(19) United States
(12) Patent Application Publication
(10) Pub. No.: US 2022/0273211 A1
(43) Pub. Date: Sep. 1, 2022

(54) FATIGUE EVALUATION SYSTEM AND FATIGUE EVALUATION DEVICE

(71) Applicant: SEMICONDUCTOR ENERGY LABORATORY CO., LTD., ATSUGI-SHI, KANAGAWA-KEN (JP)

(72) Inventors: Kengo AKIMOTO, Isehara, Kanagawa (JP); Tatsuya OKANO, Isehara, Kanagawa (JP); Motoki NAKASHIMA, Isehara, Kanagawa (JP)

(21) Appl. No.: 17/627,194

(22) PCT Filed: Jul. 20, 2020
(86) PCT No.: PCT/IB2020/056786

§ 371 Date: Jan. 14, 2022

(30) Foreign Application Priority Data
Jul. 31, 2019 (JP) 2019-141444

(51) Int. Cl. A61B 5/16

(52) U.S. Cl. CPC A61B 5/165; A61B 5/163; G06T 2207/30041

(57) ABSTRACT

A fatigue evaluation system is provided. The fatigue evaluation system includes an accumulation portion, a generation portion, a storage portion, an acquisition portion, and a measurement portion. The accumulation portion has a function of accumulating a plurality of first images and a plurality of second images. The plurality of first images are images of an eye and its surroundings acquired from a side or an oblique direction. The plurality of second images are images of an eye and its surroundings acquired from a front. The generation portion has a function of performing supervised learning and generating a learned model. The storage portion has a function of storing the learned model. The acquisition portion has a function of acquiring a third image. The third image is an image of an eye and its surroundings acquired from a side or an oblique direction. The measurement portion has a function of measuring fatigue from the third image on the basis of the learned model.

### TECHNICAL FIELD

One embodiment of the present invention relates to a fatigue evaluation method. Another embodiment of the present invention relates to a fatigue evaluation system. Another embodiment of the present invention relates to a fatigue evaluation device.

### BACKGROUND ART

In modern society, appropriate management of workers' health conditions is an important issue because it leads to not only workers' well-being but also higher labor productivity, prevention of accidents, and the like. Needless to say, appropriate management of health conditions is an important issue not only for workers but also for students, homemakers at home, and the like.

Health conditions are made worse due to fatigue accumulation. Fatigue can be classified into physical fatigue, mental fatigue, and nervous fatigue. It is comparatively easy to be aware of symptoms that appear due to physical fatigue accumulation. In contrast, in many cases, it is difficult to be aware of symptoms that appear due to mental fatigue or nervous fatigue accumulation. These days, VDT (Visual Display Terminal) work that has large visual burden has been increasing, and there are environments where nervous fatigue is likely to be accumulated.

One of the causes for fatigue can be psychological stress (also simply referred to as stress). In addition, it is said that chronic fatigue leads to disorders of autonomic nerves. Accordingly, methods for measuring fatigue or stress conditions by using machine learning or the like have attracted attention in recent years. Patent Document 1 discloses a method for detecting mental fatigue by using blinking light. Furthermore, Patent Document 2 discloses a device for evaluating an autonomic nervous function and a stress level that includes a machine learning device.

### REFERENCES

### Patent Documents

[Patent Document 1] Japanese Published Patent Application No. 2008-301841
[Patent Document 2] Japanese Published Patent Application No. 2008-259609

### SUMMARY OF THE INVENTION

### Problems to be Solved by the Invention

When fatigue or a stress level is evaluated using a detection device and the evaluation device disclosed in Patent Document 1 and Patent Document 2, in the case where a user is a worker, work related to labor needs to be interrupted; thus, labor productivity might be decreased. In addition, when the user visually recognizes the detection device, further mental fatigue might be accumulated in addition to mental fatigue accumulated before using the detection device. Accordingly, it is difficult to detect mental fatigue correctly.

In view of the above, an object of one embodiment of the present invention is to evaluate fatigue. Another embodiment of the present invention is to evaluate fatigue while suppressing the decrease in labor productivity.

Note that the description of these objects does not preclude the existence of other objects. Note that one embodiment of the present invention does not have to achieve all these objects. Note that objects other than these will be apparent from the description of the specification, the drawings, the claims, and the like, and objects other than these can be derived from the description of the specification, the drawings, the claims, and the like.

### Means for Solving the Problems

In view of the above objects, one embodiment of the present invention provides a system for evaluating fatigue (a fatigue evaluation system) on the basis of information on an eye and its surroundings acquired from a position that a user is less likely to recognize visually by generating a learned model in advance through machine learning using the information on the eye and its surroundings as learning data. Another embodiment of the present invention provides a device and an electronic device including the fatigue evaluation system.

One embodiment of the present invention is a fatigue evaluation system including an accumulation portion, a generation portion, a storage portion, an acquisition portion, and a measurement portion. The accumulation portion has a function of accumulating a plurality of first images and a plurality of second images. The plurality of first images are images of an eye and its surroundings acquired from a side or an oblique direction. The plurality of second images are images of an eye and its surroundings acquired from a front. The generation portion has a function of performing supervised learning and generating a learned model. The storage portion has a function of storing the learned model. The acquisition portion has a function of acquiring a third image. The third image is an image of an eye and its surroundings acquired from a side or an oblique direction. The measurement portion has a function of measuring fatigue from the third image on the basis of the learned model.

In the fatigue evaluation system, at least one of a pupil and a blink is preferably input for the supervised learning as training data.

In addition, in the fatigue evaluation system, one of the plurality of first images and one of the plurality of second images are preferably acquired simultaneously.

Furthermore, in the fatigue evaluation system, the side or the oblique direction is preferably at greater than or equal to 60° and less than or equal to 85° with respect to a gaze in a horizontal direction.

In addition, it is preferable that an output portion is further included in the fatigue evaluation system. Moreover, the output portion preferably has a function of providing information.

Another embodiment of the present invention is a fatigue evaluation device that includes glasses including the storage portion, the acquisition portion, and the measurement portion and a server including the accumulation portion and the generation portion in one of the fatigue evaluation systems.

### Effect of the Invention

According to one embodiment of the present invention, it is possible to evaluate fatigue. According to another embodiment of the present invention, it is possible to evaluate fatigue while suppressing the decrease in labor productivity.

Note that the effects of embodiments of the present invention are not limited to the effects listed above. The effects listed above do not preclude the existence of other effects. Note that the other effects are effects that are not described in this section and will be described below. The effects that are not described in this section can be derived from the descriptions of the specification, the drawings, and the like and can be extracted from these descriptions by those skilled in the art. Note that one embodiment of the present invention has at least one of the effects listed above and/or the other effects. Accordingly, depending on the case, one embodiment of the present invention does not have the effects listed above in some cases.

### BRIEF DESCRIPTION OF THE DRAWINGS

FIG. 1 is a diagram illustrating a structure example of a fatigue evaluation system.

FIG. 2 is a flow chart showing an example of a fatigue evaluation method.

FIG. 3A to FIG. 3C are diagrams illustrating a method for taking images of eyes and their surroundings.

FIG. 4 is a diagram illustrating a CNN structure example.

FIG. 5A and FIG. 5B are diagrams illustrating a method for taking images of eyes and their surroundings.

FIG. 6A and FIG. 6B are schematic diagrams of human's visual fields.

FIG. 7A and FIG. 7B are schematic diagrams of temporal changes in pupil diameters.

FIG. 8A and FIG. 8B are diagrams illustrating equipment and electronic devices in each of which a fatigue evaluation system is incorporated.

FIG. 9A is a diagram illustrating equipment in which part of a fatigue evaluation system is incorporated. FIG. 9B is a diagram illustrating an electronic device in which part of a fatigue evaluation system is incorporated.

### MODE FOR CARRYING OUT THE INVENTION

Embodiments will be described in detail with reference to the drawings. Note that the present invention is not limited to the following description, and it will be readily understood by those skilled in the art that modes and details of the present invention can be modified in various ways without departing from the spirit and scope of the present invention. Therefore, the present invention should not be construed as being limited to the description of embodiments below.

Note that in structures of the present invention described below, the same reference numerals are used in common for the same portions or portions having similar functions in different drawings, and a repeated description thereof is omitted. Moreover, similar functions are denoted by the same hatch pattern and are not denoted by specific reference numerals in some cases.

In addition, the position, size, range, or the like of each structure illustrated in drawings does not represent the actual position, size, range, or the like in some cases for easy understanding. Therefore, the disclosed invention is not necessarily limited to the position, size, range, or the like disclosed in the drawings.

Furthermore, ordinal numbers such as “first,” “second,” and “third” used in this specification are used in order to avoid confusion among components, and the terms do not limit the components numerically.

### Embodiment 1

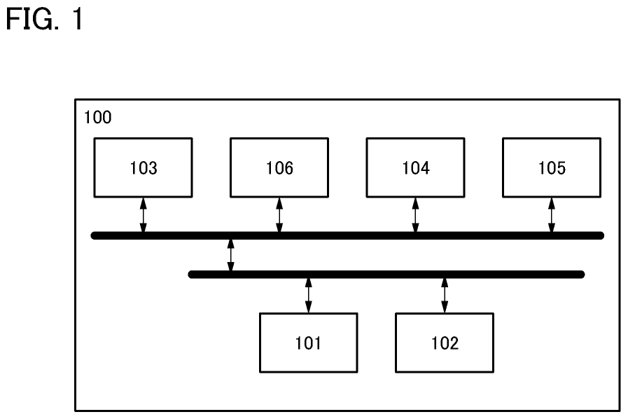

In this embodiment, a fatigue evaluation system and a fatigue evaluation method according to one embodiment of the present invention will be described using FIG. 1 to FIG. 7B.

### <Structure Example of Fatigue Evaluation System>

First, a structure example of a fatigue evaluation system is described using FIG. 1.

FIG. 1 is a diagram illustrating a structure example of a fatigue evaluation system 100. The fatigue evaluation system 100 includes an accumulation portion 101, a generation portion 102, an acquisition portion 103, a storage portion 104, a measurement portion 105, and an output portion 106.

Note that the accumulation portion 101, the generation portion 102, the acquisition portion 103, the storage portion 104, the measurement portion 105, and the output portion 106 are connected to each other through a transmission path. Note that the transmission path includes a network such as a local area network (LAN) or the Internet. In addition, for the network, wired or wireless communication or wired and wireless communication can be used.

Furthermore, in the case where a wireless communication is used for the network, besides near field communication means such as Wi-Fi (registered trademark) and Bluetooth (registered trademark), a variety of communication means such as the third generation mobile communication system (3G)-compatible communication means, LTE (sometimes also referred to as 3.9G)-compatible communication means, the fourth generation mobile communication system (4G)-compatible communication means, or the fifth generation mobile communication system (5G)-compatible communication means can be used.

Learning data is stored in the accumulation portion 101.

The generation portion 102 has a function of performing machine learning.

The acquisition portion 103 has a function of acquiring information. Here, information that is acquired by the acquisition portion 103 is information on eyes and their surroundings. For example, the acquisition portion 103 is one or more selected from a camera, a pressure sensor, a strain sensor, a temperature sensor, a gyroscope sensor, and the like.

The information that is acquired by the acquisition portion 103 is stored in the storage portion 104. A learned model is also stored in the storage portion 104.

Note that in some cases, it is not necessary to provide the storage portion 104. For example, it is not necessary to provide the storage portion 104 when the learned model and the information that is acquired by the acquisition portion 103 are stored in the accumulation portion 101.

The measurement portion 105 has a function of measuring fatigue. Note that the function of measuring fatigue includes a function of calculating the fatigue and a function of determining whether the fatigue is abnormal.

The output portion 106 has a function of providing information. The information refers to the fatigue calculated by the measurement portion 105, a result of determination of whether the fatigue is abnormal, or the like. Components included in the output portion 106 are a display, a speaker, and the like.

The above is the description of the structure example of the fatigue evaluation system 100.

### <Fatigue Evaluation Method>

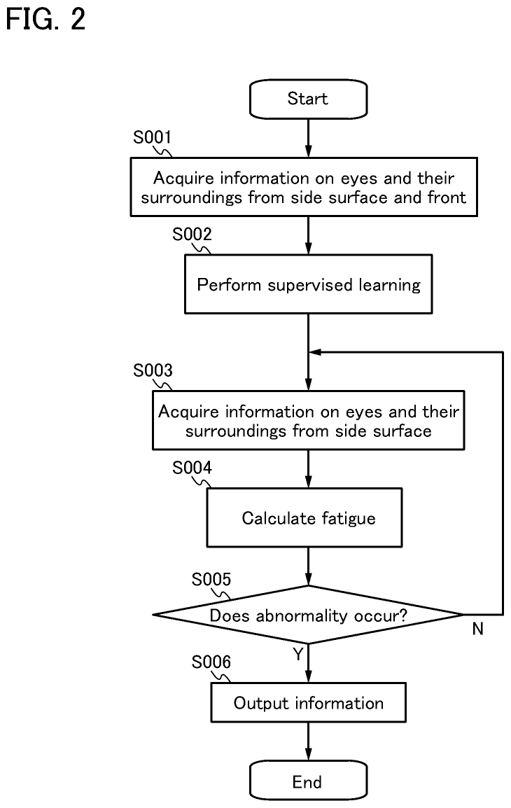

Next, examples of a fatigue evaluation method are described using FIG. 2 to FIG. 7B.

As described above, it is said that chronic fatigue leads to disorders of autonomic nerves. As the autonomic nerves, there are sympathetic nerves that become active at the time of body activity, during the daytime, and at the time of being nervous and parasympathetic nerves that become active at rest, at night, and at the time of being relaxed. When the sympathetic nerves become dominant, pupil dilation, heartbeat promotion, an increase in blood pressure, or the like occurs. In contrast, when the parasympathetic nerves become dominant, pupil contraction, heartbeat suppression, a decrease in blood pressure, or the like occurs.

When the balance of the autonomic nerves gets worse, hypothermia, a decrease in the number of blinks or the amount of tears, or the like is caused. In addition, maintaining slouching or a hunchbacked posture for a long time sometimes leads to disorders of autonomic nerves.

Accordingly, when the disorders or balance of autonomic nerves can be evaluated, fatigue can be evaluated objectively. In other words, through evaluation of temporal changes in a pupil (a pupil diameter or a pupil area), heartbeat, or a pulse, blood pressure, body temperature, a blink, posture, or the like, fatigue can be evaluated objectively.

FIG. 2 is a flow chart showing an example of a fatigue evaluation method. The fatigue evaluation method has Step S001 to Step S006 shown in FIG. 2. Step S001 and Step 002 are steps for generating a learned model, and Step S003 to Step S006 are steps for measuring fatigue.

In other words, the fatigue evaluation method includes a method for generating a learned model and a method for measuring fatigue.

### [Method for Generating Learned Model]

First, an example of the method for generating a learned model is described. The method for generating a learned model has Step S001 and Step S002 shown in FIG. 2.

In Step S001, learning data that is used to generate a learned model is prepared. For example, information on eyes and their surroundings is acquired as the learning data. That is, Step S001 can be referred to as a step of acquiring the information on eyes and their surroundings. Although described later, the information on eyes and their surroundings is preferably acquired from a side and a front, for example.

Note that the information on eyes and their surroundings is acquired using one or more selected from a camera, a pressure sensor, a strain sensor, a temperature sensor, a gyroscope sensor, and the like. Note that a publicly available data set may be used as the information on eyes and their surroundings.

For the learning data, a pupil (a pupil diameter or a pupil area), a pulse, blood pressure, body temperature, a blink, posture, a red eye, or the like is preferably provided as training data (also referred to as a training signal, a ground truth label, or the like). In particular, the pupil (the pupil diameter or the pupil area) or the blink is preferable as the training data because it is likely to change over time due to mental fatigue.

The information on eyes and their surroundings that is prepared as the learning data is accumulated in the accumulation portion 101. After the learning data is accumulated in the accumulation portion 101, the process goes to Step S002.

In Step S002, machine learning is performed based on the learning data that is accumulated in the accumulation portion 101. The machine learning is performed in the generation portion 102.

Supervised learning is preferably used for the machine learning, for example. Supervised learning utilizing a neural network (particularly, deep learning) is further preferably used for the machine learning.

For deep learning, a convolutional neural network (CNN), a recurrent neural network (RNN), an autoencoder (AE), a variational autoencoder (VAE), or the like is preferably used, for example.

A learned model is generated by the machine learning. The learned model is stored in the storage portion 104.

Note that the pupil (the pupil diameter or the pupil area), the pulse, the blood pressure, the body temperature, the blink, the posture, the red eye, or the like that is provided as the training data varies between individuals depending on age, a body shape, gender, or the like. Thus, the learned model may be updated depending on a user.

The above is the example of the method for generating a learned model.

### [Fatigue Measurement Method]

Next, an example of a fatigue measurement method is described. The fatigue measurement method has Step S003 to Step S006 shown in FIG. 2. Note that the fatigue measurement method includes a method for calculating fatigue and a method for determining whether fatigue is abnormal.

In Step S003, the information on eyes and their surroundings used for fatigue calculation is acquired.

The information on eyes and their surroundings used for fatigue calculation is preferably acquired from a side or an oblique direction, for example. When the information on eyes and their surroundings is acquired from the side or the oblique direction, the information can be acquired from a position that the user is less likely to recognize visually. Accordingly, the information can be acquired without user's awareness.

Note that the information on eyes and their surroundings used for fatigue calculation is acquired using one or more selected from a camera, a pressure sensor, a strain sensor, a temperature sensor, a gyroscope sensor, and the like.

In addition, the information on eyes and their surroundings used for fatigue calculation is acquired in chronological order.

The information on eyes and their surroundings used for fatigue calculation is stored in the storage portion 104. After the information is stored in the storage portion 104, the process goes to Step S004.

In Step S004, fatigue is calculated. The learned model generated in Step S002 and the information on eyes and their surroundings acquired in Step S003 are used for fatigue calculation. Note that fatigue is calculated in the measurement portion 105.

Fatigue calculation refers to numerical conversion of an index for evaluating fatigue. As the index for evaluating fatigue, at least one of the pupil (the pupil diameter or the pupil area), the pulse, the blood pressure, the body temperature, the blink, the posture, the red eye, and the like is used, for example.

Note that fatigue calculation is not limited to numerical conversion of the index for evaluating fatigue. For example, fatigue may be numerically converted from the information on eyes and their surroundings acquired in Step S003 by using the learned model.

Note that before the process goes to Step S005, Step S003 and Step S004 are repeated for a certain period. Accordingly, chronological data for determining whether abnormality occurs in the index for evaluating fatigue can be acquired.

In Step S005, whether abnormality occurs in the index for evaluating fatigue is determined.

In the case where it is determined that abnormality occurs in the index for evaluating fatigue, it is determined that the level of fatigue is high. In the case where it is determined that the level of fatigue is high, the process goes to Step S006. In contrast, in the case where it is determined that abnormality does not occur in the index for evaluating fatigue, it is determined that the level of fatigue is not high. In the case where it is determined that the level of fatigue is not high, the process goes to Step S003.

Note that in Step S004, in the case where fatigue is converted into a numerical value, whether abnormality occurs in the numerical value of fatigue is determined. In the case where it is determined that abnormality occurs in the numerical value of fatigue, it is determined that the level of fatigue is high. In the case where it is determined that the level of fatigue is high, the process goes to Step S006. In contrast, in the case where it is determined that abnormality does not occur in the numerical value of fatigue, it is determined that the level of fatigue is not high. In the case where it is determined that the level of fatigue is not high, the process goes to Step S003.

In Step S006, information is output. The information refers to the index for evaluating fatigue calculated by the measurement portion 105, fatigue converted into a numerical value, a result of determination of whether fatigue is abnormal, and the like. The information is output as, for example, visual information such as a character string, a numerical value, a graph, or a color, audio information such as a voice or music, or the like.

After the information is output, the process is terminated.

The above is the description of the example of the method for calculating fatigue.

The above is the description of the example of the fatigue evaluation method.

### <<Specific Example of Fatigue Evaluation Method>>

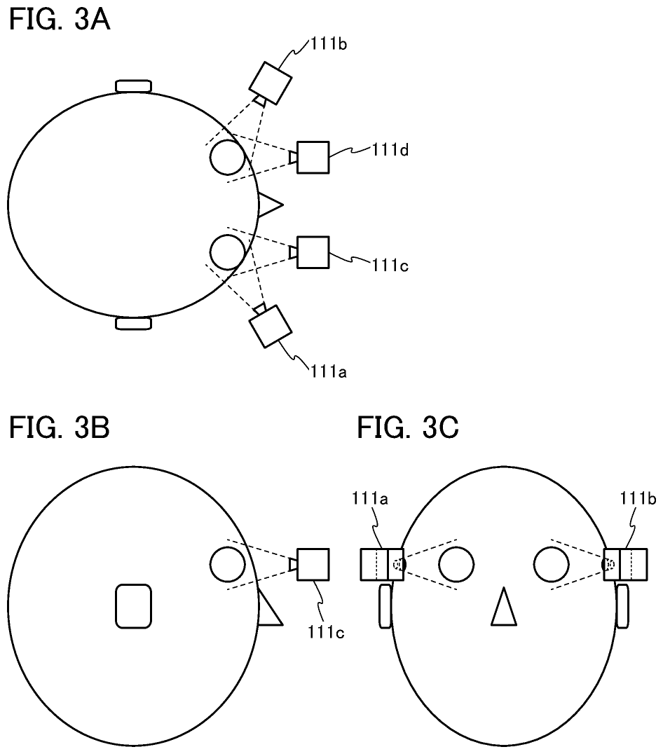

In this section, specific examples of a fatigue evaluation method are described using FIG. 3A to FIG. 7B. Here, a temporal change in the pupil (the pupil diameter or the pupil area) is selected as the index for evaluating fatigue.

Image data of eyes and their surroundings is used as the learning data prepared in Step S001, for example. At this time, the image data of eyes and their surroundings are preferably image data of eyes and their surroundings acquired from the front and image data of eyes and their surroundings acquired from the side or the oblique direction. As compared to the image data acquired from the side or the oblique direction, in the image data acquired from the front, the pupil (the pupil diameter or the pupil area) can be detected with high accuracy. Accordingly, as compared to when the image data acquired from the side or the oblique direction is only used as the learning data, when the image data acquired from the front and the image data acquired from the side or the oblique direction are used as the learning data, a highly accurate learned model can be generated.

Note that images of eyes and their surroundings for the learning data are preferably acquired by taking images from the front and the side or the oblique direction using a camera or the like, for example.

Examples of taking images from the front and the side or the oblique direction using a camera 111a to a camera 111d are illustrated in FIG. 3A to FIG. 3C. FIG. 3A is a diagram of a target person for photography seen from above. FIG. 3B is a diagram of the target person for photography seen from a right side. FIG. 3C is a diagram of the target person for photography seen from the front. Note that for clarity of the diagrams, the camera 111a, the camera 111b, and the camera 111d are omitted in FIG. 3B, and the camera 111c and the camera 111d are omitted in FIG. 3C. Note that the target person for photography is not necessarily limited to a person (user) whose fatigue is evaluated.

As illustrated in FIG. 3A to FIG. 3C, images of eyes and their surroundings are taken from the front using the camera 111c and the camera 111d. In addition, images of eyes and their surroundings are taken from the side or the oblique direction using the camera 111a and the camera 111b.

Before the machine learning is performed, processing or correction of image data for the learning data may be performed. Examples of processing or correction of image data include cutting of portions that are not required for the machine learning, grayscale conversion, a median filter, a Gaussian filter, and the like. Processing or correction of image data can reduce noise generated in the machine learning.

A plurality of combinations of image data of eyes and their surroundings acquired from the front and image data of eyes and their surroundings acquired from the side or the oblique direction that are acquired simultaneously are preferably prepared as the learning data. When images of eyes and their surroundings are taken from a plurality of angles simultaneously, the processing or correction can be facilitated. Accordingly, a highly accurate learned model can be generated. For example, in consideration of the image data of eyes and their surroundings acquired from the front, processing or correction of the image data of eyes and their surroundings acquired from the side or the oblique direction is performed. Thus, outlines of pupils in the image data of eyes and their surroundings acquired from the side or the oblique direction are emphasized, and the pupils (pupil diameters or pupil areas) can be detected with high accuracy.

Note that in the case where a lot of images can be acquired from the side or the oblique direction, only the image data acquired from the side or the oblique direction may be used as the learning data. In addition, in the case where processing or correction of the image data of eyes and their surroundings acquired from the side or the oblique direction is performed in consideration of the image data of eyes and their surroundings acquired from the front, only the image data acquired from the side or the oblique direction may be used as the learning data.

Since the learning data is the image data as described above, a convolutional neural network is preferably used for the machine learning performed in Step S002.

### [Convolutional Neural Network]

Here, a convolutional neural network (CNN) is described.

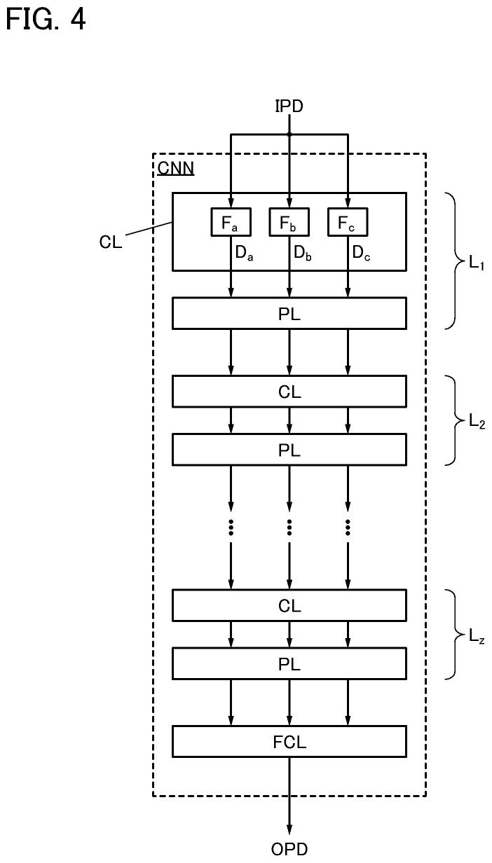

FIG. 4 illustrates a CNN structure example. The CNN is formed of a convolution layer CL, a pooling layer PL, and a fully connected layer FCL. Image data IPD is input to the CNN, and feature extraction is performed. In this embodiment, the image data IPD is image data of eyes and their surroundings.

The convolution layer CL has a function of performing convolution on image data. The convolution is performed by repetition of the product-sum operation of part of the image data and a filter value of a weight filter (also referred to as a kernel). By the convolution in the convolution layer CL, a feature of an image is extracted.

The product-sum operation may be performed using a program on software or may be performed by hardware. In the case where the product-sum operation is performed by hardware, a product-sum operation circuit can be used. A digital circuit may be used or an analog circuit may be used as this product-sum operation circuit.

The product-sum operation circuit may be formed using a transistor including Si in a channel formation region (also referred to as a Si transistor) or may be formed using a transistor including a metal oxide in a channel formation region (also referred to as an OS transistor). An OS transistor is particularly suitable for a transistor included in an analog memory of the product-sum operation circuit because of its extremely low off-state current. Note that the product-sum operation circuit may be formed using both a Si transistor and an OS transistor.

For the convolution, one or a plurality of weight filters can be used. In the case of using a plurality of weight filters, a plurality of features of the image data can be extracted. FIG. 4 illustrates an example in which three filters (filters Fₐ, F_{b}, and F_{c}) are used as weight filters. The image data input to the convolution layer CL is subjected to filter processing using the filters Fₐ, F_{b}, and F_{c}, so that image data Dₐ, d_{b}, and D_{c} are generated. Note that the image data Dₐ, D_{b}, and D_{c} are also referred to as feature maps.

The image data Dₐ, D_{b}, and D_{c} generated by the convolution are converted using an activation function and then output to the pooling layer PL. As the activation function, ReLU (Rectified Linear Units) or the like can be used. ReLU is a function that outputs “0” when an input value is negative and outputs the input value as it is when the input value is greater than or equal to “0.” As the activation function, a sigmoid function, a tanh function, or the like can also be used.

The pooling layer PL has a function of performing pooling on the image data input from the convolution layer CL. Pooling is processing in which the image data is partitioned into a plurality of regions, predetermined data is extracted from each of the regions, and the data are arranged in a matrix. By the pooling, the spatial size of the image data is shrunk while the features extracted by the convolution layer CL remain. In addition, the position invariance or movement invariance of the features extracted by the convolution layer CL can be increased. Note that as the pooling, max pooling, average pooling, Lp pooling, or the like can be used.

In the CNN, feature extraction is performed using the convolution processing and pooling processing. Note that the CNN can be composed of a plurality of convolution layers CL and a plurality of pooling layers PL. FIG. 4 illustrates a structure in which z layers L (z is an integer greater than or equal to 1) each including the convolution layer CL and the pooling layer PL are provided (a layer L₁ to a layer L_{z}) and the convolution processing and the pooling processing are performed z times. In this case, feature extraction can be performed in each layer L, which enables more advanced feature extraction.

The fully connected layer FCL has a function of determining an image using the image data subjected to convolution and pooling. All nodes in the fully connected layer FCL are connected to all nodes in a layer prior to the fully connected layer FCL (here, the pooling layer PL or the pooling layer PL included in the layer L_{Z} in FIG. 4). Image data output from the convolution layer CL or the pooling layer PL is a two-dimensional feature map and is unfolded into a one-dimensional feature map when input to the fully connected layer FCL. Then, data OPD that is unfolded one-dimensionally is output.

Note that the structure of the CNN is not limited to the structure in FIG. 4. For example, the pooling layer PL may be provided for a plurality of convolutional layers CL. Moreover, in the case where the positional information of the extracted feature is desired to be left as much as possible, the pooling layer PL may be omitted.

Furthermore, in the case of classifying images using output data from the fully connected layer FCL, an output layer electrically connected to the fully connected layer FCL may be provided. The output layer can output probability of classification into each class using a softmax function or the like as a likelihood function. Classification classes are preferably the levels of fatigue, for example. Specifically, the classification classes are “the level of fatigue is extremely high,” “the level of fatigue is high,” “the level of fatigue is moderate,” “the level of fatigue is low,” “the level of fatigue is extremely low,” and the like. Accordingly, fatigue can be converted into numerical values from image data.

Furthermore, in the case of performing regression analysis such as numerical value prediction from the output data of the fully connected layer FCL, an output layer electrically connected to the fully connected layer FCL may be provided. The use of an identity function or the like for the output layer enables output of a predicted value. Accordingly, a pupil diameter or a pupil area can be calculated from the image data, for example.

In addition, the CNN can perform supervised learning using image data as learning data to which training data is added. In the supervised learning, a backpropagation method can be used, for example. Owing to the learning in the CNN, the filter value of the weight filter, the weight coefficient of the fully connected layer, or the like can be optimized.

The above is the description of the convolutional neural network (CNN).

In the supervised learning, image data of eyes and their surroundings acquired from the front and image data of eyes and their surroundings acquired from the side or the oblique direction are prepared as the learning data, and learning is performed such that the pupil diameter or the pupil area is output. For example, in the case where the pupil diameter or the pupil area is input as the training data, the pupil diameter or the pupil area is output owing to regression using the CNN. Through the above process, a learned model for outputting the pupil diameter or the pupil area is generated from the image data of eyes and their surroundings acquired from the side or the oblique direction.

Note that fatigue converted into a numerical value may be output by class classification using the CNN. At this time, a learned model for outputting fatigue converted into a numerical value is generated from the image data of eyes and their surroundings acquired from the side or the oblique direction.

In Step S003, information on eyes and their surroundings used for fatigue calculation is acquired. Images of eyes and their surroundings that are acquired from the side or the oblique direction are acquired as the information on eyes and their surroundings, for example. Note that the images of eyes and their surroundings are preferably acquired by taking images from the side using a camera or the like.

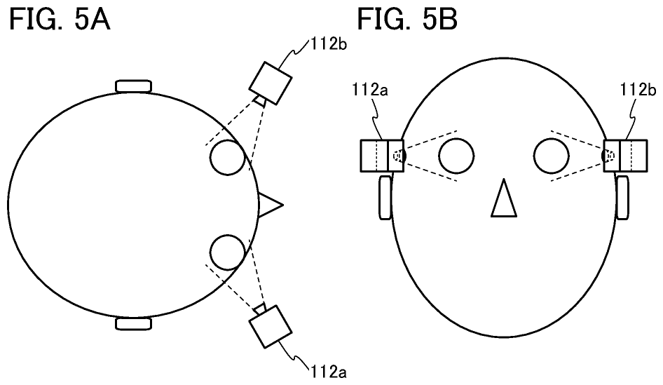

Examples of taking images from the side or the oblique direction using a camera 112a and a camera 112b are illustrated in FIG. 5A and FIG. 5B. FIG. 5A is a diagram of a target person for photography seen from above. FIG. 5B is a diagram of the target person for photography seen from the front. Note that the target person for photography is a person (user) whose fatigue is evaluated.

As illustrated in FIG. 5A and FIG. 5B, images of eyes and their surroundings are taken from the side or the oblique direction using the camera 112a and the camera 112b.

Note that the distance from a camera 111 (either one or more of the camera 111a to the camera 111d) illustrated in FIG. 3A to a subject of photography and the distance from a camera 112 (the camera 112a and/or the camera 112b) illustrated in FIG. 5A to the subject of photography are preferably substantially equal to each other. This enables fatigue evaluation with high accuracy. Note that since supervised learning is performed in the fatigue evaluation method according to one embodiment of the present invention, the distances to the subject of photography need not necessarily be equal to each other in the camera 111 and the camera 112.

In addition, an image taken using the camera 111 and an image taken using the camera 112 preferably have the same resolution, aspect ratio, and the like. This enables fatigue evaluation with high accuracy. Note that the supervised learning is performed in the fatigue evaluation method according to one embodiment of the present invention; therefore, the image taken using the camera 111 and the image taken using the camera 112 need not necessarily have the same resolution, aspect ratio, and the like.

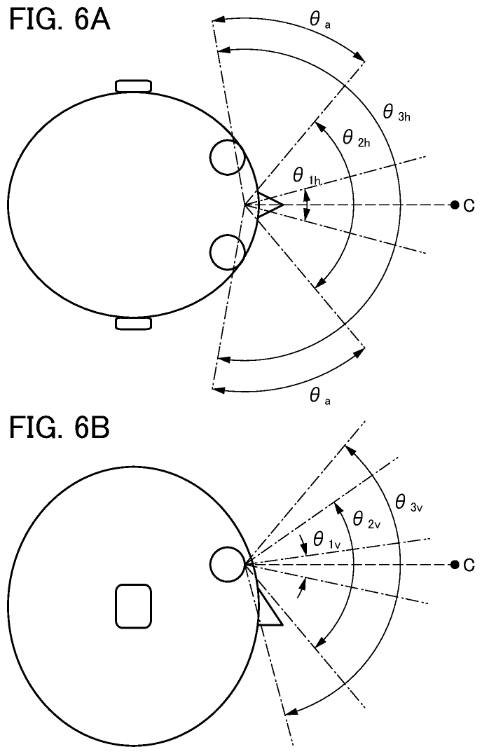

FIG. 6A and FIG. 6B illustrate schematic diagrams of a human's visual field (binocular vision). FIG. 6A is a diagram of a person seen from above. FIG. 6B is a diagram of a person seen from a right side.

The human's visual field is classified into an effective visual field, an induced visual field, an auxiliary visual field, and the like. In FIG. 6A and FIG. 6B, a line from a person to a gaze point C that is shown by a broken line is gaze (a visual axis); an angle Din and an angle θ₁ᵥ correspond to a viewing angle range of the effective visual field; an angle θ₂ₕ and an angle θ₂ᵥ correspond to a viewing angle range of the induced visual field; and an angle θ₃ₕ and an angle θ_{3V} correspond to a viewing angle range of the auxiliary visual field. Note that unless otherwise specified, the gaze refers to a line from the person to the gaze point C when the gaze point exists at a position where the length of a line segment that connects the gaze point C and the right eye is equal to the length of a line segment that connects the gaze point C and the left eye. In addition, a horizontal direction refers to a direction horizontal to a plane including both of the eyes and the gaze. Furthermore, a perpendicular direction refers to a direction perpendicular to the plane including both of the eyes and the gaze.

The effective visual field is a region where information can be received instantaneously. Note that it is said that a viewing angle of the effective visual field in the horizontal direction (the angle θ₁ₕ illustrated in FIG. 6A) is a range of approximately 30° with the gaze used as a center, and it is said that a viewing angle of the effective visual field in the perpendicular direction (the angle θ₁ᵥ illustrated in FIG. 6B) is a range of approximately 20° with a portion slightly below the gaze used as a center.

The induced visual field is a region that affects a spatial coordinate system. Note that it is said that a viewing angle of the induced visual field in the horizontal direction (the angle θ₂ₕ illustrated in FIG. 6A) is a range of approximately 100° with the gaze used as a center, and it is said that a viewing angle of the induced visual field in the perpendicular direction (the angle θ₂ᵥ illustrated in FIG. 6B) is a range of approximately 85° with the portion slightly below the gaze used as a center.

The auxiliary visual field is a region where the presence of a stimulus can be perceived. Note that it is said that a viewing angle of the auxiliary visual field in the horizontal direction (the angle θ₃ₕ illustrated in FIG. 6A) is a range of approximately 200° with the gaze used as a center, and it is said that a viewing angle of the auxiliary visual field in the perpendicular direction (the angle θ_{3y} illustrated in FIG. 6B) is a range of approximately 125° with the portion slightly below the gaze used as a center.

Information during work is received most from the effective visual field and is also received slightly from the induced visual field. In addition, there is almost no information from the auxiliary visual field during the work. In other words, a worker is less likely to recognize information located in the auxiliary visual field.

In addition, in the case where a temporal change in the pupil is selected as the index for evaluating fatigue, the pupil needs to be included in the image acquired in Step S003. Visual information is recognized when an image projected on a retina through the pupil, a crystalline lens, and the like is transmitted to a brain via optic nerves. In other words, since the auxiliary visual field also includes the visual information, the pupil can be recognized in the auxiliary visual field.

Accordingly, the side or the oblique direction from which the images of eyes and their surroundings are acquired is a horizontal direction where the pupil is observed from inside the auxiliary visual field or the inside of the induced visual field in the vicinity of the auxiliary visual field. The side or the oblique direction is within the range of an angle θₐ illustrated in FIG. 6A. Specifically, the side or the oblique direction refers to at greater than or equal to 45° and less than or equal to 100°, preferably greater than or equal to 50° and less than or equal to 90°, further preferably greater than or equal to 60° and less than or equal to 85° in a horizontal direction with respect to the gaze. Therefore, the images can be acquired from a position that is less likely to be visually recognized by the user. Therefore, the images can be acquired without user's awareness.

Note that in the case where the side or the oblique direction is within the above range in the horizontal direction, either angle is out of the viewing angle of the induced visual field in the perpendicular direction. Thus, the side or the oblique direction in the perpendicular direction may be any direction as long as it is within the range where images of the pupil can be taken.

In addition, the front from which the images of eyes and their surroundings are acquired is a horizontal direction where the pupil is observed from inside the induced visual field. Specifically, the front is within a range of greater than or equal to 0° and less than or equal to 50°, preferably greater than or equal to 0° and less than or equal to 30°, further preferably greater than or equal to 0° and less than or equal to 15° in a horizontal direction with respect to the gaze. Accordingly, images of a pupil with a circle or circle-like shape can be taken, and a pupil diameter or a pupil area can be calculated with high accuracy.

In the case where the temporal change in the pupil is selected as the index for evaluating fatigue, the pupil diameter or the pupil area is calculated from the image data of eyes and their surroundings acquired from the side or the oblique direction using the learned model in Step S004.

As described above, the pupil dilates when sympathetic nerves become dominant, and the pupil contracts when parasympathetic nerves become dominant. In other words, the pupil diameter changes in accordance with disorders of autonomic nerves. In addition, it is said that change speed of the pupil becomes slow when fatigue accumulates. In this specification, a temporal change in a pupil (a pupil diameter or a pupil area) refers to a time change in a pupil (a pupil diameter or a pupil area), change speed of a pupil (a pupil diameter or a pupil area), a time change in the expansion and contraction cycle of a pupil (a pupil diameter or a pupil area), or the like.

Whether abnormality occurs in the temporal change in the pupil (the pupil diameter or the pupil area) is determined with reference to the pupil (the pupil diameter or the pupil area) immediately after start of Step S003.

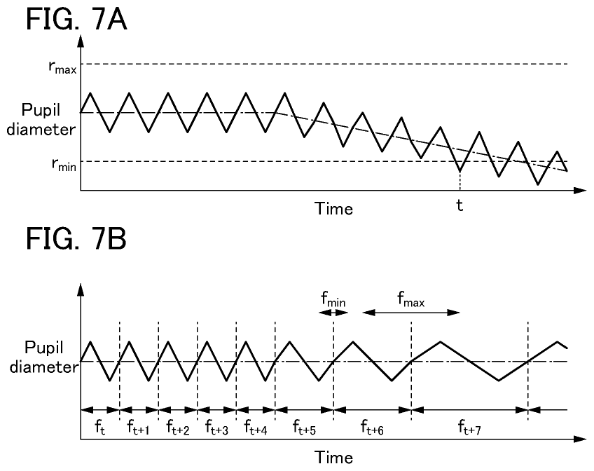

An example of a method for determining whether abnormality occurs in the temporal change in the pupil (the pupil diameter or the pupil area) is described using FIG. 7A and FIG. 7B.

FIG. 7A and FIG. 7B are schematic diagrams of temporal changes in pupil diameters. In FIG. 7A and FIG. 7B, horizontal axes each represent time, and vertical axes each represent a pupil diameter. Solid lines in FIG. 7A and FIG. 7B represent temporal changes in the pupil diameters. In addition, dashed-dotted lines in FIG. 7A and FIG. 7B represent time averages of the pupil diameters.

FIG. 7A is a diagram schematically illustrating a state where the pupil diameter decreases over time. In order to determine whether the temporal change in the pupil diameter is abnormal, a threshold value of the pupil diameter is set in advance. For example, as illustrated in broken lines in FIG. 7A, the upper limit of the pupil diameter is set to rₘₐₓ, and the lower limit of the pupil diameter is set to rₘᵢₙ. In the example of FIG. 7A, the pupil diameter at time t is smaller than the lower limit rₘᵢₙ of the pupil diameter. At this time, it is determined that abnormality occurs in the temporal change in the pupil.

For example, in the case where the pupil (the pupil diameter or the pupil area) dilates or contracts at a certain rate or higher with reference to the pupil (the pupil diameter or the pupil area) immediately after start of Step S003, it is determined that abnormality occurs.

Furthermore, FIG. 7B is a diagram schematically illustrating a state where the expansion and contraction cycle of the pupil diameter extends over time. The expansion and contraction cycle of the pupil diameter is set to fᵤ (u is a natural number). In order to determine whether the temporal change in the pupil diameter is abnormal, a threshold value of the expansion and contraction cycle of the pupil diameter is set in advance. For example, as illustrated in FIG. 7B, the upper limit of the expansion and contraction cycle of the pupil diameter is set to fₘₐₓ, and the lower limit of the expansion and contraction cycle of the pupil diameter is set to fₘᵢₙ. In the example of FIG. 7B, the expansion and contraction cycle of the pupil diameter fₜ₊₇ is larger than the upper limit of the expansion and contraction cycle of the pupil diameter fₘₐₓ. At this time, it is determined that abnormality occurs in the temporal change in the pupil.

Note that mixture of expansion and contraction of the pupil diameter and the expansion and contraction cycle of the pupil diameter is observed. For example, fast Fourier transform may be performed on the temporal change in the pupil diameter. This facilitates determining whether abnormality occurs with reference to the expansion and contraction cycle of the pupil diameter.

Note that although an example of a method for calculating the pupil diameter or the pupil area from the image data of eyes and their surroundings acquired from the side or the oblique direction by using the learned model is illustrated, the present invention is not limited to this. For example, fatigue may be converted into a numerical value from the image data of eyes and their surroundings acquired from the side or the oblique direction by using the learned model. At this time, in order to determine whether fatigue converted into a numerical value is abnormal, a threshold value of fatigue (the upper limit of fatigue) is set in advance.

The above is the description of the fatigue evaluation system. With the use of the fatigue evaluation system according to one embodiment of the present invention, the system (in particular, the acquisition portion) is not positioned on a user's gaze; thus, an increase in user's mental fatigue is suppressed. This enables highly accurate evaluation of fatigue during use.

The structure, method, and the like described in this embodiment can be used in an appropriate combination with the structure, the method, and the like described in the other embodiment and the like.

### Embodiment 2

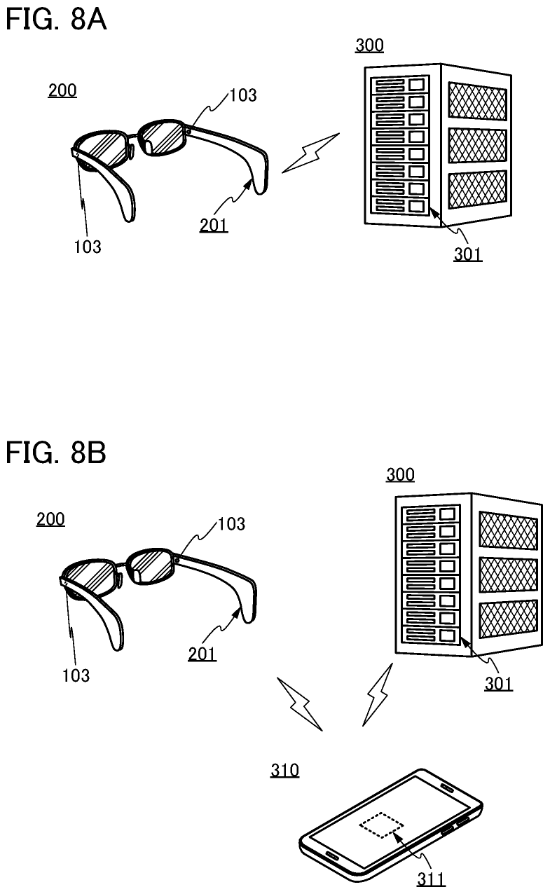

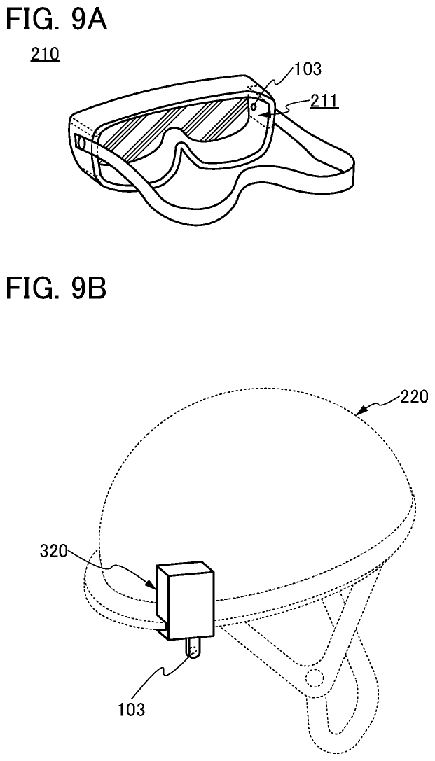

In this embodiment, fatigue evaluation devices are described using FIG. 8A to FIG. 9B. The fatigue evaluation devices are electronic devices or equipment and an electronic device including the fatigue evaluation system described in the above embodiment.

Examples of equipment including part of the fatigue evaluation system include glasses such as vision correction glasses and safety glasses, a safety protector that is to be mounted on a head, such as a helmet and a gas mask, and the like.

The equipment includes at least the acquisition portion 103 in the fatigue evaluation system described in the above embodiment. In addition, the equipment includes a battery.

Examples of an electronic device including part of the fatigue evaluation system include an information terminal, a computer, and the like. Note that here, examples of the computer include not only a tablet computer, a laptop computer, and a desktop computer but also a large computer such as a work station and a server system.

Note that when a GPS (Global Positioning System) receiver is mounted on the equipment, the electronic device may acquire data on the position, travel distance, acceleration, or the like of the equipment using a GPS. A combination of the acquired data with the index for evaluating fatigue enables fatigue evaluation with higher accuracy.

FIG. 8A illustrates examples of the equipment and the electronic device including the fatigue evaluation system. FIG. 8A are glasses 200 and a server 300 including the fatigue evaluation system. The glasses 200 include a processing portion 201. In addition, the server 300 includes a processing portion 301.

For example, the processing portion 201 includes the acquisition portion 103 that is described in the above embodiment, and the processing portion 301 includes the accumulation portion 101, the generation portion 102, the storage portion 104, and the measurement portion 105 that are described in the above embodiment. In addition, the processing portion 201 and the processing portion 301 each include a transmission/reception portion. Since the processing portion 201 includes only the acquisition portion 103, weight reduction of the glasses 200 that include the processing portion 201 can be achieved. Accordingly, user's physical burden when wearing the glasses 200 can be reduced.

In addition, in the case where a camera is used as the acquisition portion 103, placing the acquisition portion 103 in the vicinities of eyes in frames of the glasses 200 makes it possible to take close-up images of eyes and their surroundings. This facilitates eye detection. In addition, outside scenery reflections in eyes can be reduced. Thus, the number of times of processing or correction of images of eyes and their surroundings can be reduced. Alternatively, processing or correction becomes unnecessary.

Note that although FIG. 8A illustrates the example in which the camera is used as the acquisition portion 103, the present invention is not limited to this. A pressure sensor, a strain sensor, a temperature sensor, a gyroscope sensor, or the like may be used as the acquisition portion 103. At this time, the acquisition portion 103 may be provided at a position other than the side or the oblique direction of the eye. For example, the acquisition portion 103 may be provided at a portion where the head is in contact with the frame of the glasses 200 or the vicinity thereof.

Note that the processing portion 201 may include the output portion 106 described in the above embodiment. When the processing portion 201 includes the output portion 106, the user can know fatigue during the work. Components included in the output portion 106 are a display, a speaker, and the like.

Note that information provided from the output portion 106 is preferably output as visual information such as a color, audio information such as a voice or music, or the like. Compared to visual information such as a character string, a numerical value, or a graph, visual information such as a color is preferable because influence on vision is small and stress on the user is low. The same applies to audio information such as a voice or music. Note that when favorite music or the like is registered in advance as the audio information, user's fatigue can be sometimes reduced.

Note that the structures of the processing portion 201 and the processing portion 301 are not limited thereto. For example, the processing portion 201 may include the acquisition portion 103, the storage portion 104, the measurement portion 105, and the output portion 106, and the processing portion 301 may include the accumulation portion 101 and the generation portion 102. At this time, the processing portion 201 has a function of measuring fatigue, and the processing portion 301 has a function of generating a learned model.

With the above structures, fatigue can be measured by only the processing portion 201; therefore, the frequency of communication between the processing portion 201 and the processing portion 301 can be kept to the minimum. In addition, with the above structures, a learned model updated by the processing portion 301 can be transmitted from the processing portion 301 to the processing portion 201, and the processing portion 201 can receive the learned model. Then, the learned model stored in the processing portion 201 can be updated to the received learned model. Accordingly, learning data with improved accuracy can be utilized, and fatigue can be evaluated with higher accuracy.

The information on eyes and their surroundings that is acquired by the acquisition portion 103 may be accumulated in the storage portion 104. After a certain amount of the acquired information on eyes and their surroundings is accumulated in the storage portion 104, the accumulated information may be transmitted to the electronic device including the processing portion 301. Accordingly, the number of times of communication between the processing portion 201 and the processing portion 301 can be reduced.

Note that a plurality of electronic devices including part of the fatigue evaluation system may be formed. FIG. 8B illustrates glasses, a server, and a cellular phone (a smartphone), which is a kind of information terminal, including the fatigue evaluation system. Like the glasses 200 and the server 300 illustrated in FIG. 8A, the glasses 200 include the processing portion 201, and the server 300 includes the processing portion 301. In addition, an information terminal 310 includes a processing portion 311.

For example, the processing portion 201 includes the acquisition portion 103. The processing portion 301 includes the accumulation portion 101 and the generation portion 102. The processing portion 311 includes the storage portion 104, the measurement portion 105, and the output portion 106. In addition, the processing portion 201, the processing portion 301, and the processing portion 311 each include a transmission/reception portion.

In the above structures, in the case where the information terminal 310 is owned by the user of the glasses 200, the user can confirm his or her fatigue through the information terminal 310.

In addition, in the case where the information terminal 310 is owned by a boss or the like of the user of the glasses 200, the boss or the like of the user can confirm the user's fatigue through the information terminal 310. Thus, even in the case where the user and the user's boss are not close to each other, the user's boss can monitor user's health conditions. Furthermore, in the case where information output from the output portion 106 is visual information on fatigue, such as a character string, a numerical value, or a graph, details of the user's health conditions can be known.

The glasses 200 illustrated in FIG. 8A and FIG. 8B are not limited to vision correction glasses, and may be sunglasses, color vision correction glasses, 3D glasses, augmented reality (AR) glasses, mixed reality (MR) glasses, plain glasses, glasses for a personal computer with a bluelight cutting function, or the like.

In particular, when information on fatigue is output as visual information such as a character string, a numerical value, or a graph in the AR glasses, the MR glasses, or the like, details of fatigue can be known.

FIG. 9A illustrates safety glasses including part of the fatigue evaluation system. Safety glasses 210 illustrated in FIG. 9A include a processing portion 211. In addition, the processing portion 211 includes the acquisition portion 103.

The processing portion 211 preferably has a function similar to that of the processing portion 201 included in the glasses 200 illustrated in FIG. 8A and FIG. 8B.

Note that although FIG. 9A illustrates goggle type safety glasses as the safety glasses 210, the present invention is not limited thereto. The safety glasses 210 may be spectacle type safety glasses or front type safety glasses. In addition, although FIG. 9A illustrates single lens type safety glasses, the present invention is not limited thereto. The safety glasses 210 may be twin-lens type safety glasses.

Although FIG. 8A, FIG. 8B, and FIG. 9A illustrate glasses such as vision correction glasses and safety glasses as the equipment including part of the fatigue evaluation system, the present invention is not limited thereto. Examples of the equipment including part of the fatigue evaluation system include a safety protector that is to be mounted on a head, such as a helmet and a gas mask.

Although the structure where the equipment including part of the fatigue evaluation system is combined with the electronic device including part of the fatigue evaluation system is described as the fatigue evaluation device so far, the present invention is not limited thereto. The fatigue evaluation device may have a structure where a detachable electronic device including part of the fatigue evaluation system is combined with the above electronic device, for example. FIG. 9B illustrates a safety protector 220 to be mounted on the head where a detachable electronic device 320 including part of the fatigue evaluation system is attached. The detachable electronic device 320 includes the acquisition portion 103. When the detachable electronic device 320 includes the part of the fatigue evaluation system, a safety protector that has been conventionally used can be utilized.

In addition, the part of the fatigue evaluation system may be included in a display device that is to be mounted on the head, such as a head-mounted display or smart glasses, for example. Accordingly, fatigue can be evaluated even when virtual reality (VR) is utilized, for example.

Note that the fatigue evaluation device may be single equipment or a single electronic device including the fatigue evaluation system.

With the use of the fatigue evaluation device according to one embodiment of the present invention, user's visibility is secured and user's mental burden is reduced. This enables evaluation of user's fatigue with high accuracy. In addition, in the case where the user is a worker, the use of the fatigue evaluation device according to one embodiment of the present invention eliminates the need to interrupt the work for fatigue evaluation; thus, a decrease in labor productivity can be suppressed.

In addition, with the fatigue evaluation device according to one embodiment of the present invention, information on eyes and their surroundings can be acquired from a position that is close to the eyes. Accordingly, fatigue evaluation accuracy can be increased.

The structure, method, and the like described in this embodiment can be used in an appropriate combination with the structure, the method, and the like described in the other embodiment and the like.

### REFERENCE NUMERALS

100: fatigue evaluation system, 101: accumulation portion, 102: generation portion, 103: acquisition portion, 104: storage portion, 105: measurement portion, 106: output portion, 111: camera, 111a: camera, 111b: camera, 111c: camera, 111d: camera, 112: camera, 112a: camera, 112b: camera, 200: glasses, 201: processing portion, 210: safety glasses, 211: processing portion, 220: safety protector, 300: server, 301: processing portion, 310: information terminal, 311: processing portion, and 320: electronic device.

1. A fatigue evaluation system comprising:
an accumulation portion,
a generation portion,
a storage portion,
an acquisition portion, and
a measurement portion,
wherein the accumulation portion is configured to accumulate a plurality of first images and a plurality of second images,
wherein the plurality of first images are images of an eye and its surroundings acquired from a side or an oblique direction,
wherein the plurality of second images are images of an eye and its surroundings acquired from a front,
wherein the generation portion is configured to perform supervised learning and generate a learned model,
wherein the storage portion is configured to store the learned model,
wherein the acquisition portion is configured to acquire a third image,
wherein the third image is an image of an eye and its surroundings acquired from a side or an oblique direction, and
wherein the measurement portion is configured to measure fatigue from the third image on the basis of the learned model.

2. The fatigue evaluation system according to claim 1, wherein data on at least one of a pupil and a blink is input for the supervised learning as training data.

3. The fatigue evaluation system according to claim 1, wherein one of the plurality of first images and one of the plurality of second images are acquired simultaneously.

4. The fatigue evaluation system according to claim 1, wherein the side or the oblique direction is at greater than or equal to 60° and less than or equal to 85° with respect to a gaze in a horizontal direction.

5. The fatigue evaluation system according to claim 1, further comprising an output portion,
wherein the output portion is configured to provide information on the fatigue and a result of determination of whether the fatigue is abnormal or not.

6. A fatigue evaluation device comprising glasses including the storage portion, the acquisition portion, and the measurement portion and a server including the accumulation portion and the generation portion in one of the fatigue evaluation systems according to claim 1.
